(19) United States
(12) Patent Application Publication
(10) Pub. No.: US 2025/0104821 A1
(43) Pub. Date: Mar. 27, 2025

(54) HEALTH INFORMATION OUTPUT APPARATUS, HEALTH INFORMATION OUTPUT METHOD, AND NON-TRANSITORY COMPUTER-READABLE MEDIUM

(71) Applicant: NEC Corporation, Minato-ku, Tokyo (JP)

(72) Inventor: Chieko Kuribayashi, Tokyo (JP)

(73) Assignee: NEC Corporation, Minato-ku, Tokyo (JP)

(21) Appl. No.: 18/832,258

(22) PCT Filed: Feb. 3, 2022
(86) PCT No.: PCT/JP2022/004279

§ 371 Date: Jul. 23, 2024

(51) Int. Cl. G16H 10/60

(52) U.S. Cl. CPC G16H 10/60

(57) ABSTRACT

A health information storage unit (20) stores, for each of a plurality of individuals, health information of the individual, and individual identification information identifying the individual in association with each other. A relative information storage unit (30) stores relative information. The relative information is information being capable of determining, for each of a plurality of individuals, individual identification information of a relative of the individual. A acquisition unit (110) acquires individual identification information of a target person. A determination unit (120) determines the individual identification information of a relative of the target person by using the relative information storage unit (30). A output unit (130) acquires, from the health information storage unit (20), the health information associated with the individual identification information determined by the determination unit (120), and outputs the acquired health information.

### TECHNICAL FIELD

The present invention relates to a health information output apparatus, a health information output method, and a program.

### BACKGROUND ART

In recent years, digitizing a medical record is in progress. Further, in recent years, digitizing information written in a maternal and child health handbook has also been considered. For example, Patent Document 1 describes recording, in an IC card, health information relating to a mother and child written in a maternal and child health handbook, and also storing, in the IC card, link information for linking the health information to medical information stored in a medical information database of each medical institution. Further, Patent Document 1 also describes that health information also includes family identification information identifying a parent and a sibling of a child, and that health information of the child and medical information relating to the parent and the sibling of the child are linked to each other, based on the family identification information.

### RELATED DOCUMENT

### Patent Document

Patent Document 1: Japanese Patent Application Publication No. 2000-285178

### DISCLOSURE OF THE INVENTION

### Technical Problem

According to Patent Document 1, a person in question being a child, and medical information of a parent and a sibling of the person in question can be linked to each other. However, Patent Document 1 does not disclose how to acquire family identification information of the parent and the sibling. Therefore, in the technique described in Patent Document 1, there has been a possibility that effort is required when family identification information of a parent and a sibling is determined.

In view of the above-described problem, one example of an object of the present invention is to provide a health information output apparatus, a health information output method, and a program that enable to reduce effort required when health information of a relative is enabled to be acquired.

### Solution to Problem

One aspect of the present invention provides a health information output apparatus being capable of utilizing a first storage unit that stores, for each of a plurality of individuals, health information of the individual, and individual identification information identifying the individual in association with each other, and being capable of utilizing a second storage unit that stores, for each of a plurality of the individuals, relative information being capable of determining the individual identification information of a relative of the individual, including:
an acquisition unit that acquires the individual identification information of a target person;
a determination unit that determines the individual identification information of a relative of the target person by using the second storage unit; and
an output unit that acquires, from the first storage unit, the health information associated with the individual identification information determined by the determination unit, and outputs the acquired health information.

One aspect of the present invention provides a health information output method to be performed by a computer, the method including,
by the computer;
   being capable of utilizing a first storage unit that stores, for each of a plurality of individuals, health information of the individual, and individual identification information identifying the individual in association with each other, and being capable of utilizing a second storage unit that stores, for each of a plurality of the individuals, relative information being capable of determining the individual identification information of a relative of the individual;
   acquiring the individual identification information of a target person;
   determining the individual identification information of a relative of the target person by using the second storage unit; and
   acquiring, from the first storage unit, the health information associated with the individual identification information determined by the determination unit, and outputting the acquired health information.

One aspect of the present invention provides a program being executable by a computer, wherein
the computer is capable of utilizing a first storage unit that stores, for each of a plurality of individuals, health information of the individual, and individual identification information identifying the individual in association with each other, and is capable of utilizing a second storage unit that stores, for each of a plurality of the individuals, relative information being capable of determining the individual identification information of a relative of the individual,
the program causing the computer to include:
   an acquisition function of acquiring the individual identification information of a target person;
   a determination function of determining the individual identification information of a relative of the target person by using the second storage unit; and
   an output function of acquiring, from the first storage unit, the health information associated with the individual identification information determined by the determination unit, and outputting the acquired health information.

### Advantageous Effects of Invention

One aspect of the present invention provides a health information output apparatus, a health information output method, and a program that enable to reduce effort required when health information of a relative is enabled to be acquired.

### BRIEF DESCRIPTION OF THE DRAWINGS

FIG. 1 is a diagram illustrating an overview of a health information output apparatus according to an example embodiment.

FIG. 2 is a diagram illustrating a detail example of a usage environment of the health information output apparatus together with a configuration of the health information output apparatus.

FIG. 3 is a diagram illustrating one example of information stored in a first sub storage unit.

FIG. 4 is a diagram illustrating one example of information stored in a second sub storage unit.

FIG. 5 is a diagram illustrating one example of information stored in a relative information storage unit.

FIG. 6 is a diagram illustrating a first example of information stored in a range information storage unit.

FIG. 7 is a diagram illustrating a second example of information stored in the range information storage unit.

FIG. 8 is a diagram illustrating a hardware configuration example of the health information output apparatus.

FIG. 9 is a flowchart illustrating a first example of processing to be performed by the health information output apparatus.

FIG. 10 is a flowchart illustrating a second example of processing to be performed by the health information output apparatus.

FIG. 11 is a flowchart illustrating a third example of processing to be performed by the health information output apparatus.

### EXAMPLE EMBODIMENT

Hereinafter, an example embodiment according to the present invention is described by using the drawings. Note that, in all drawings, a similar constituent element is indicated by a similar reference sign, and description thereof is omitted as necessary.

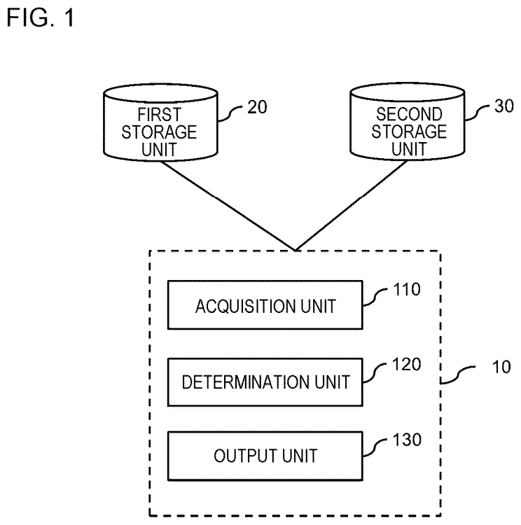

FIG. 1 is a diagram illustrating an overview of a health information output apparatus 10 according to the example embodiment. The health information output apparatus 10 can utilize a health information storage unit 20 and a relative information storage unit 30, and includes an acquisition unit 110, a determination unit 120, and an output unit 130.

The health information storage unit 20 stores, for each of a plurality of individuals, health information of the individual, and individual identification information identifying the individual in association with each other. The relative information storage unit 30 stores, for each of a plurality of individuals, relative information being capable of determining individual identification information of a relative of the individual.

The acquisition unit 110 acquires individual identification information of a target person. The determination unit 120 determines individual identification information of a relative of the target person by using the relative information storage unit 30. The output unit 130 acquires, from the health information storage unit 20, health information associated with the individual identification information determined by the determination unit 120, and outputs the acquired health information. The health information is health information of the relative of the target person.

The health information output apparatus 10 can utilize the relative information storage unit 30. As described above, the relative information storage unit 30 stores, for each of a plurality of individuals, relative information being capable of determining individual identification information of a relative of the individual. Therefore, it is possible to reduce effort required when health information of a relative of a target person is enabled to be acquired.

Hereinafter, a detail example of the health information output apparatus 10 is described.

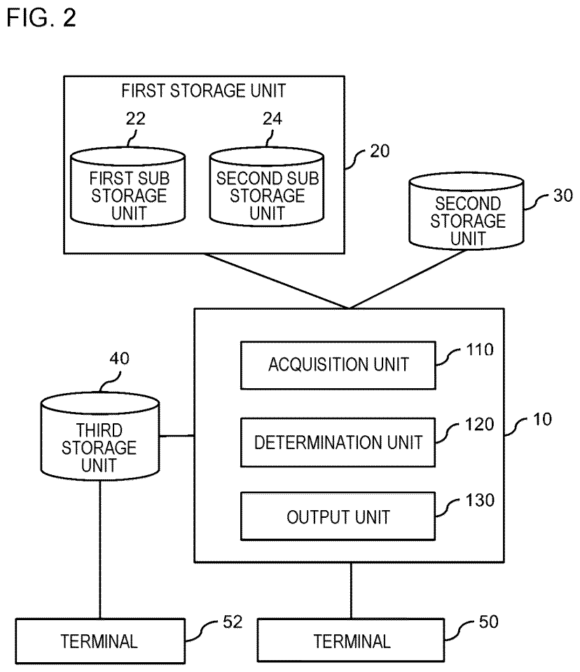

FIG. 2 is a diagram illustrating a detail example of a usage environment of the health information output apparatus 10 together with a configuration of the health information output apparatus 10. The health information output apparatus 10 is utilized when health information of a relative of a target person is disclosed. The health information output apparatus 10 is used together with a range information storage unit 40 and terminals 50 and 52, in addition to the health information storage unit 20 and the relative information storage unit 30 illustrated in FIG. 1.

The health information storage unit 20 stores, for each of a plurality of individuals, health information of the individual, and individual identification information identifying the individual in association with each other. The individual identification information is, for example, an individual number, but may be information other than the above. The health information includes, for example, medical record information to be generated when the individual utilizes a medical institution, and birth information generated when the individual is born.

The medical record information is, for example, medical information, and includes information indicating a treatment progress in a medical institution. One example of the medical record information is at least one of a medical history, an inspection record, a nursing record, and a medication history, and the medical record information is input by a medical personnel such as a doctor.

The birth information is, for example, information written in a maternal and child health handbook, and includes, for example, at least one of progress information when the individual is a fetus, progress information when the individual is born, and progress information when the individual is an infant. Herein, an upper limit of an age of an “infant” is, for example, an upper limit of an age to be managed by a maternal and child health handbook, for example, 6 years old, but the age is not limited thereto.

Note that, health information of a woman who has given birth may include birth information of a child born to the woman.

In the example illustrated in FIG. 2, the health information storage unit 20 includes a first sub storage unit 22 and a second sub storage unit 24. The first sub storage unit 22 stores, for each of a plurality of individuals, medical record information in association with individual identification information. The second sub storage unit 24 stores, for each of a plurality of individuals, birth information in association with individual identification information. The first sub storage unit 22 and the second sub storage unit 24 may be servers different from each other, or may be a same server. In the former case, an administrator of a server including the first sub storage unit 22, and an administrator of a server including the second sub storage unit 24 may be different from each other. Further, the first sub storage unit 22 may be provided for each of a plurality of medical institutions.

The relative information storage unit 30 stores relative information for each of a plurality of individuals. The relative information is information being capable of determining individual identification information of a relative of the individual. The relative information may include, for example, individual identification information of a relative, or may include a name of a relative. In the latter case, individual identification information of a relative is determined by using a database in which a name of an individual and individual identification information are associated with each other. The database may be the relative information storage unit 30 itself. The relative information storage unit 30 preferably stores information being capable of determining a relative having at least a blood relation and within a second degree of kinship.

The relative information storage unit 30 may be, for example, a database managing family register information, or may be a database other than the database. Further, one example of timing at which the relative information storage unit 30 is updated is, for example, timing at which a birth registration is submitted to a local government. The birth registration includes a name of a newborn baby, and a name of his/her parents. Therefore, using information written in a birth registration enables to provides a newborn baby with individual identification information, and enables to store, in the relative information storage unit 30, the individual identification information in association with individual identification information of the parents of the newborn baby.

The range information storage unit 40 stores disclosed relative information and disclosure range information.

The disclosed relative information is information determining a relative whose health information can be disclosed. One example of a relative to be determined by the disclosed relative information is at least one of a parent, a grandparent, a child, a grandchild, and a sibling. When a relationship between a target person and an individual is out of a range of the disclosed relative information, even when the individual is a relative of the target person, health information of the individual is not disclosed.

The disclosure range information indicates a range of health information disclosable to a relative. When the disclosure range information is set, health information of an individual is disclosed within a range of the disclosure range information.

Note that, the disclosed relative information may be set for each individual, or may be set as common information among a plurality of individuals. Likewise, the disclosure range information may be set for each individual, or may be set as common information among a plurality of individuals. When the disclosed relative information and the disclosure range information are set for each individual, these pieces of information are stored in association with individual identification information. Note that, regarding at least one individual, at least one of the disclosed relative information and the disclosure range information is not needed to be set.

In the example illustrated in FIG. 2, the range information storage unit 40 is located outside the health information output apparatus 10. However, the range information storage unit 40 may be a part of the health information output apparatus 10.

The terminal 50 is operated by a person who wishes to browse health information of a relative of a target person, namely, a browsing applicant. The browsing applicant transmits individual identification information of the target person to the health information output apparatus 10 via the terminal 50. Then, the health information output apparatus 10 determines a relative of the target person, and transmits health information of the relative to the terminal 50. The browsing applicant may be, for example, the target person himself/herself, or may be a medical personnel.

The terminal 52 is used when information stored in the range information storage unit 40 is updated. An individual who wishes to set disclosed relative information sets the disclosed relative information by operating the terminal 52. Likewise, an individual who wishes to set disclosure range information operates the disclosure range information by operating the terminal 52. In any case, the individual inputs individual identification information of the individual to the terminal 52, and updates disclosed relative information (or disclosure range information) associated with the individual identification information in the range information storage unit 40.

Note that, the terminal 52 may be, for example, a mobile terminal such as a smartphone, or may be a fixed terminal. Further, the terminals 50 and 52 may be a same terminal. Next, a detail example of a function of the health information output apparatus 10 is described. The health information output apparatus 10 includes the acquisition unit 110, the determination unit 120, and the output unit 130.

As described by using FIG. 1, the acquisition unit 110 acquires individual identification information of a person in which health information of a relative is required, namely, a target person. Herein, a person who wishes to browse health information of a relative, namely, a browsing applicant may be, as described above, the target person himself/herself, or may be a medical personnel. In the example illustrated in FIG. 1, the acquisition unit 110 acquires individual identification information of the target person from the terminal 50.

As described by using FIG. 1, the determination unit 120 determines individual identification information of a relative of a target person by using the relative information storage unit 30. As one example, the determination unit 120 reads, from the relative information storage unit 30, individual identification information associated with individual identification information acquired by the acquisition unit 110. The individual identification information read herein is individual identification information of a relative of the target person.

When the range information storage unit 40 stores disclosed relative information, the determination unit 120 defines a range of relatives determining individual identification information according to the disclosed relative information. As described above, the disclosed relative information is information determining a relative whose health information can be disclosed. In other words, the determination unit 120 determines individual identification information of a relative within a predetermined range. One example of the predetermined range is at least one of a parent, a grandparent, a child, a grandchild, and a sibling.

The disclosed relative information may indicate a range of relatives when a target person is set as a reference. In this case, the determination unit 120 acquires, from among relatives of the target person, individual identification information of a relative indicated by the disclosed relative information. As one example, the determination unit 120 acquires individual identification information of at least one of a parent, a grandparent, a child, a grandchild, and a sibling of the target person. This example is preferable, for example, when disclosed relative information is set commonly to a plurality of individuals. In this case, the disclosed relative information is updated, for example, by an administrator of the health information output apparatus 10.

Further, the disclosed relative information may indicate a range of relatives when a relative is set as a reference, specifically; up to which relative, health information of the person in question is allowed to be disclosed when the range is viewed from the person in question. In this case, the determination unit 120 determines, from among the relatives, a relative in which the target person is included in disclosed relative information associated with the relative, and acquires individual identification information of the determined relative. As one example, when a “child” is included in disclosed relative information of a parent of the target person, the determination unit 120 determines individual identification information of the parent of the target person. On the other hand, when a “child” is not included in disclosed relative information of a parent of the target person, the determination unit 120 does not determine individual identification information of the parent of the target person. Likewise, when a “sibling” is included in disclosed relative information of a sibling of the target person, the determination unit 120 determines individual identification information of the sibling of the target person. Further, when a “sibling” is not included in disclosed relative information of a sibling of the target person, the determination unit 120 does not determine individual identification information of the sibling of the target person. This example is preferable, for example, when disclosed relative information is set for each of a plurality of pieces of individual identification information. In this case, the disclosed relative information is updated, for example, by an individual associated with the disclosed relative information.

The output unit 130 acquires, from the health information storage unit 20, health information associated with individual identification information determined by the determination unit 120, and outputs the acquired health information. The health information is health information of a relative of the target person.

When the range information storage unit 40 stores disclosure range information, the output unit 130 defines a range of health information to be output according to the disclosure range information. As a first example, the output unit 130 acquires, from the health information storage unit 20, health information within a range indicated by disclosure range information from among health information associated with individual identification information determined by the determination unit 120. As a second example, the output unit 130 may read all pieces of health information associated with individual identification information determined by the determination unit 120, and then, select and output, to the terminal 50, health information within a range indicated by disclosure range information. Note that, when disclosure range information is set for each individual, the output unit 130 reads, from the range information storage unit 40, disclosure range information associated with individual identification information acquired by the determination unit 120, and uses the read disclosure range information.

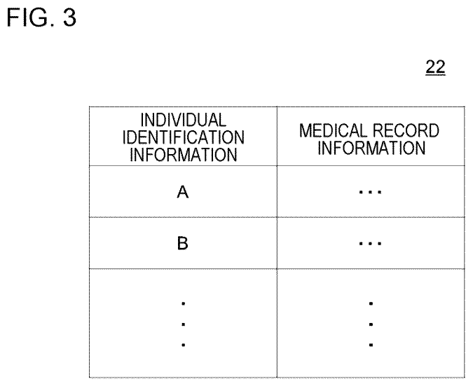

FIG. 3 is a diagram illustrating one example of information stored in the first sub storage unit 22 illustrated in FIG. 2. The first sub storage unit 22 stores medical record information of an individual in association with individual identification information of the individual. The medical record information is generated, for example, by a medical institution, however, the first sub storage unit 22 may be provided for each of a plurality of medical institutions, or may be provided as a common storage unit among a plurality of medical institutions.

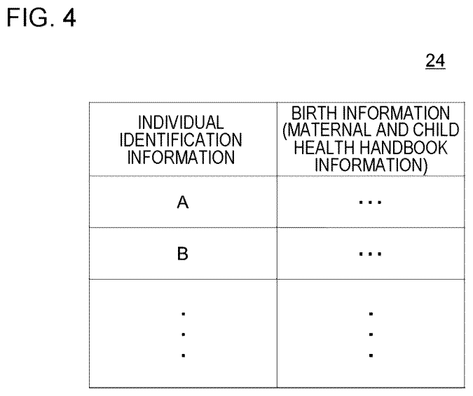

FIG. 4 is a diagram illustrating one example of information stored in the second sub storge unit 24 illustrated in FIG. 2. The second sub storage unit 24 stores birth information of an individual in association with individual identification information of the individual. The birth information is, as described by using FIG. 2, for example, information to be written in a maternal and child health handbook, and includes at least one of progress information when the individual is a fetus, progress information when the individual is born, and progress information when the individual is an infant. The “progress information when the individual is a fetus” is generated by a medical institution, for example, during a time when a mother of the individual is pregnant with the individual. The “progress information when the individual is born” is generated by a medical institution, for example, when a mother of the individual gave birth to the individual. The “progress information when the individual is an infant” is generated by a medical institution, for example, when the individual is an infant. Note that, there is a case where a part of birth information is identical to a part of medical record information.

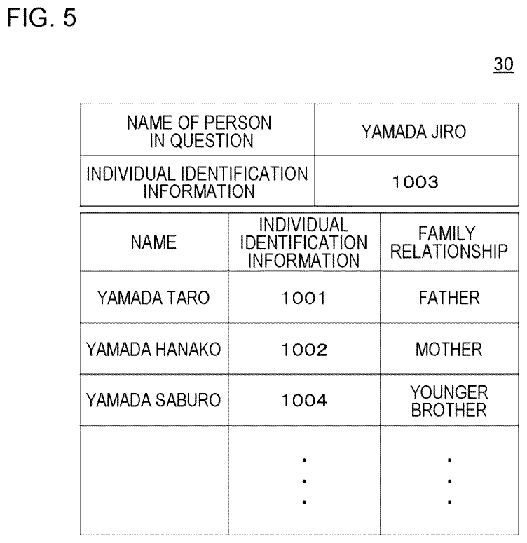

FIG. 5 is a diagram illustrating one example of information stored in the relative information storage unit 30 illustrated in FIG. 2. The relative information storage unit 30 stores relative information for each of a plurality of individuals. In the example illustrated in FIG. 5, the relative information storage unit 30 stores, for each of a plurality of individuals, a name and individual identification information of the individual, and detail information of each relative. The detail information of a relative includes a name and individual identification information of the relative, and a family relationship of the relative when the individual is set as a reference.

Preferably, the relative information storage unit 30 may store relative information at least up to a second degree of kinship. The relative information storage unit 30 may further store relative information of a third degree of kinship or higher.

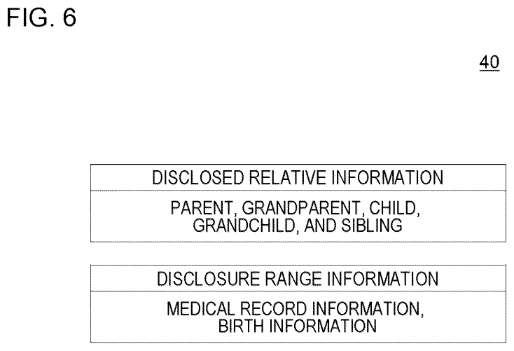

FIG. 6 is a diagram illustrating a first example of information stored in the range information storage unit 40 illustrated in FIG. 2. The range information storage unit 40 stores disclosed relative information and disclosure range information. In the example illustrated in FIG. 6, both of the disclosed relative information and the disclosure range information become information to be applied commonly to all individuals.

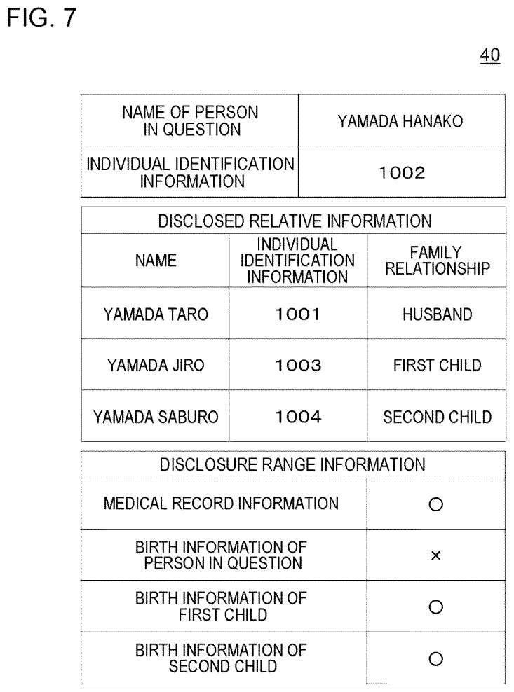

FIG. 7 is a diagram illustrating a second example of information stored in the range information storage unit 40 illustrated in FIG. 2. In the example illustrated in FIG. 7, disclosed relative information and disclosure range information are stored for each of a plurality of individuals. Specifically, the range information storage unit 40 stores a name and individual identification information of the individual (person in question), disclosed relative information, and disclosure range information in association with one another. The disclosed relative information includes information determining a relative to whom health information of the person in question can be disclosed, for example, at least one of a name, individual identification information, and a family relationship. Further, the disclosure range information indicates, for each item (e.g., medical record information, and birth information of the person in question) of health information, whether the item is allowed to be disclosed to a relative. Further, when the person in question is a woman who has given birth, an item of health information includes birth information of a child born to the woman.

Note that, the range information storage unit 40 may store disclosed relative information, as information to be applied commonly to all individuals, and store disclosure range information for each of a plurality of individuals. Further, the range information storage unit 40 may store disclosed relative information for each of a plurality of individuals, and store disclosure range information, as information to be applied commonly to all individuals.

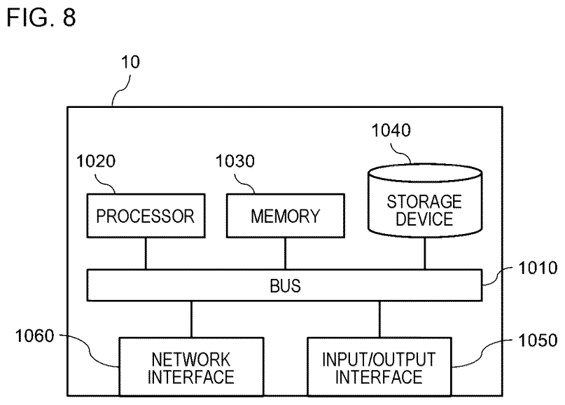

FIG. 8 is a diagram illustrating a hardware configuration example of the health information output apparatus 10. The health information output apparatus 10 includes a bus 1010, a processor 1020, a memory 1030, a storage device 1040, an input/output interface 1050, and a network interface 1060.

The bus 1010 is a data transmission path along which the processor 1020, the memory 1030, the storage device 1040, the input/output interface 1050, and the network interface 1060 mutually transmit and receive data. However, a method of mutually connecting the processor 1020 and the like is not limited to bus connection.

The processor 1020 is a processor to be achieved by a central processing unit (CPU), a graphics processing unit (GPU), or the like.

The memory 1030 is a main storage apparatus to be achieved by a random access memory (RAM) or the like.

The storage device 1040 is an auxiliary storage apparatus to be achieved by a hard disk drive (HDD), a solid state drive (SSD), a removable medium such as a memory card, a read only memory (ROM), or the like. The storage device 1040 stores a program module achieving each function (e.g., the acquisition unit 110, the determination unit 120, and the output unit 130) of the health information output apparatus 10. Each function associated with a program module is achieved by causing the processor 1020 to read each program module in the memory 1030 and execute the program module. Further, when the range information storage unit 40 is a part of the health information output apparatus 10, the storage device 1040 also functions as the range information storage unit 40.

The input/output interface 1050 is an interface for connecting the health information output apparatus 10 to various pieces of input/output equipment.

The network interface 1060 is an interface for connecting the health information output apparatus 10 to a network. The network is, for example, a local area network (LAN) or a wide area network (WAN). A method of connecting the network interface 1060 to the network may be wireless connection, or may be wired connection. The health information output apparatus 10 may communicate with the health information storage unit 20, the relative information storage unit 30, the range information storage unit 40, and the terminal 50 via the network interface 1060.

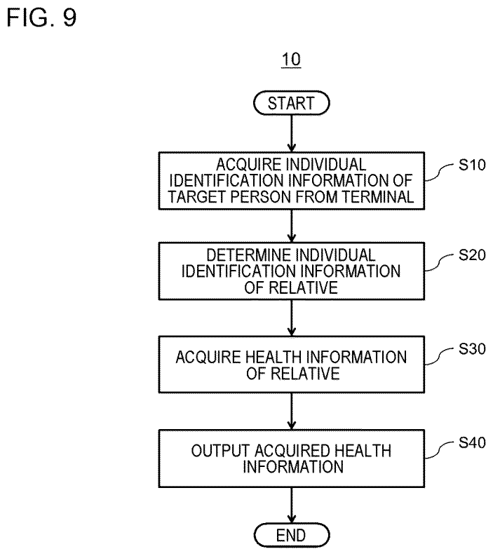

FIG. 9 is a flowchart illustrating a first example of processing to be performed by the health information output apparatus 10. First, a person who wishes to browse health information of a relative of a target person, namely, a browsing applicant operates the terminal 50, and transmits individual identification information of the target person to the health information output apparatus 10. The acquisition unit 110 of the health information output apparatus 10 acquires the individual identification information (step S10).

Subsequently, the determination unit 120 of the health information output apparatus 10 acquires relative information of the target person by using the relative information storage unit 30, and determines individual identification information of a relative of the target person by using the relative information (step S20).

Subsequently, the output unit 130 of the health information output apparatus 10 acquires, from the health information storage unit 20, health information associated with the individual identification information determined in step S20. The health information is health information of the relative of the target person (step S30).

Subsequently, the output unit 130 of the health information output apparatus 10 outputs the acquired health information to the terminal 50 (step S40). The terminal 50 displays the health information. In this way, the browsing applicant can confirm health information of the relative of the target person.

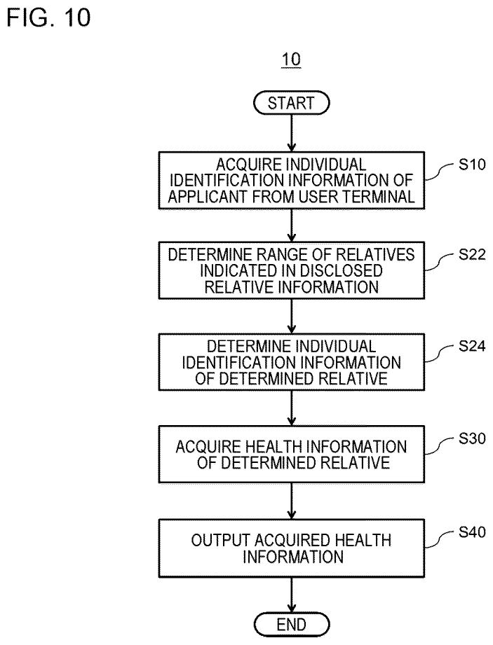

FIG. 10 is a flowchart illustrating a second example of processing to be performed by the health information output apparatus 10. The example illustrated in FIG. 10 is similar to the example illustrated in FIG. 9 except for a point that disclosed relative information is used.

First, the acquisition unit 110 of the health information output apparatus 10 acquires individual identification information of a target person similarly to the example illustrated in FIG. 9 (step S10).

Subsequently, the determination unit 120 of the health information output apparatus 10 acquires, from the range information storage unit 40, disclosed relative information, and determines a range of relatives indicated by the disclosed relative information (step S22). Subsequently, the determination unit 120 acquires, from the relative information storage unit 30, individual identification information of a relative included in the range (step S24).

For example, when the disclosed relative information indicates a range of relatives when a target person is set as a reference, the determination unit 120 acquires disclosed relative information of the target person, and recognizes a range of relatives indicated by the disclosed relative information. Then, individual identification information of a relative (e.g., a parent, and a sibling when viewed from the target person) included in the range is acquired from the relative information storage unit 30.

On the other hand, when the disclosed relative information indicates a range of relatives when a relative is set as a reference, first, the determination unit 120 determines individual identification information of a relative of the target person by using the relative information storage unit 30, and acquires disclosure range information associated with the individual identification information, specifically, disclosure range information of a relative. Then, when the target person is included in the disclosure range information, individual identification information of the relative is added to the individual identification information serving as a disclosure target of health information.

As one example, when a “child” is included in disclosure range information associated with a parent of the target person, the determination unit 120 adds individual identification information of the parent to individual identification information serving as a disclosure target of health information. On the other hand, when a “child” is not included in disclosure range information associated with a parent of the target person, the determination unit 120 does not add individual identification information of the parent to individual identification information serving as a disclosure target of health information.

The determination unit 120 performs the processing with respect to a plurality of relatives.

Pieces of processing (steps S30 and S40) thereafter are similar to the example illustrated in FIG. 9.

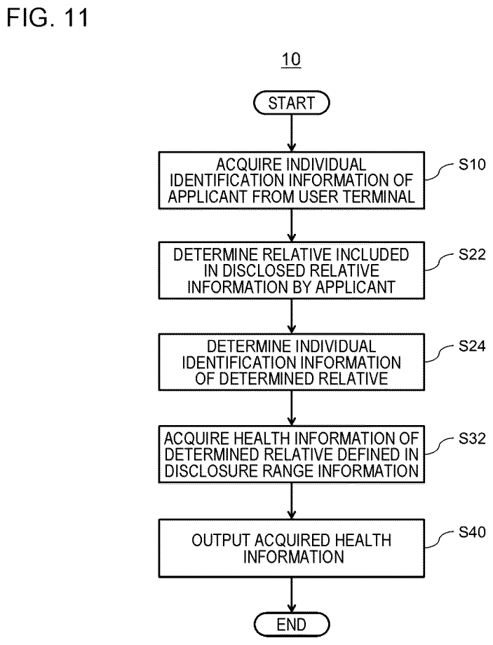

FIG. 11 is a flowchart illustrating a third example of processing to be performed by the health information output apparatus 10. The example illustrated in FIG. 11 is similar to the example illustrated in FIG. 9 except for a point that disclosure range information is used.

Pieces of processing from step S10 to step S24 are similar to the example illustrated in FIG. 9. Then, the output unit 130 selects and acquires an item defined in the disclosure range information from among the health information (step S32).

As one example, when disclosure range information indicates that only medical record information is allowed to be disclosed, the output unit 130 acquires only medical record information of a relative.

Further, when disclosure range information is set for each relative, the output unit 130 selects an item that should be output to the terminal 50 for each relative. As one example, when disclosure range information of a parent indicates that only medical record information is allowed to be disclosed, the output unit 130 acquires only medical record information, as health information of the parent. Further, when disclosure range information of a sibling indicates that both of medical record information and birth information are allowed to be disclosed, the output unit 130 selects the medical record information and the birth information, as health information of the sibling.

Then, the output unit 130 outputs the acquired health information to the terminal 50 (step S40).

As described above, according to the present example embodiment, the health information output apparatus 10 includes the determination unit 120 and the output unit 130. The determination unit 120 acquires individual identification information of a relative of a target person by using individual identification information of the target person and the relative information storage unit 30. Then, the output unit 130 reads, from the health information storage unit 20, health information associated with the individual identification information acquired by the determination unit 120, and outputs the read health information to the terminal 50. Therefore, effort required when health information of a relative is enabled to be acquired can be reduced.

Further, regarding some of relatives stored in the relative information storage unit 30, there is a case where it is better not to allow a browsing applicant to browse health information. In this case, the health information output apparatus 10 uses disclosed relative information stored in the range information storage unit 40. Specifically, the determination unit 120 or the output unit 130 limits a range of relatives whose health information should be output according to disclosed relative information. This enables to limit a range of relatives whose health information is output.

Further, there is a case where it is better not to allow a browsing applicant to browse some items of health information. In this case, the health information output apparatus 10 uses disclosure range information stored in the range information storage unit 40. Specifically, the output unit 130 limits an item to be output to the terminal 50 among health information according to disclosure range information. This enables to limit a range of health information to be output to the terminal 50.

In the foregoing, an example embodiment according to the present invention has been described with reference to the drawings, however, these are examples of the present invention, and various configurations other than the above can also be adopted.

Further, in a plurality of flowcharts used in the above description, a plurality of processes (pieces of processing) are described in order, however, an order of execution of processes to be performed in each example embodiment is not limited to the order of description. In each example embodiment, the illustrated order of processes can be changed within a range that does not adversely affect a content. Further, the above-described each example embodiment can be combined, as far as contents do not conflict with each other.

A part or all of the above-described example embodiment may also be described as the following supplementary notes, but is not limited to the following.

1. A health information output apparatus being capable of utilizing a first storage unit that stores, for each of a plurality of individuals, health information of the individual, and individual identification information identifying the individual in association with each other, and being capable of utilizing a second storage unit that stores, for each of a plurality of the individuals, relative information being capable of determining the individual identification information of a relative of the individual, including:
an acquisition unit that acquires the individual identification information of a target person;
a determination unit that determines the individual identification information of a relative of the target person by using the second storage unit; and
an output unit that acquires, from the first storage unit, the health information associated with the individual identification information determined by the determination unit, and outputs the acquired health information.

2. The health information output apparatus according to supplementary note 1, wherein
disclosed relative information that determines a relative being capable of disclosing the health information of the individual is defined for each piece of the individual identification information of a plurality of the individuals, and
the output unit determines the disclosed relative information associated with the individual identification information determined by the determination unit, and outputs the health information, when the target person is included in the disclosed relative information.

3. The health information output apparatus according to supplementary note 1 or 2, wherein the determination unit determines the individual identification information of the relative within a predetermined range.

4. The health information output apparatus according to supplementary note 3, wherein the relative within the predetermined range is at least one of a parent, a grandparent, a child, a grandchild, and a sibling.

5. The health information output apparatus according to any one of supplementary notes 1 to 4,wherein
disclosure range information indicating a range of the health information disclosable to a relative is defined, and
the output unit defines a range of the health information to be output according to the disclosure range information.

6. The health information output apparatus according to supplementary note 5, wherein
a disclosure range of the health information associated with the individual identification information is defined for each of a plurality of pieces of the individual identification information.

7. The health information output apparatus according to any one of supplementary notes 1 to 6,wherein
the health information includes birth information including at least one of progress information when the individual is a fetus, progress information when the individual is born, and progress information when the individual is an infant.

8. The health information output apparatus according to supplementary note 7, wherein
the first storage unit includes a first sub storage unit that stores medical record information of the individual, and a second sub storage unit that stores the birth information.

9. The health information output apparatus according to any one of supplementary notes 1 to 8,wherein
the second storage unit is updated according to a birth registration.

10. A health information output method to be performed by a computer, the method including,
by the computer;
   being capable of utilizing a first storage unit that stores, for each of a plurality of individuals, health information of the individual, and individual identification information identifying the individual in association with each other, and being capable of utilizing a second storage unit that stores, for each of a plurality of the individuals, relative information being capable of determining the individual identification information of a relative of the individual;
   acquiring the individual identification information of a target person;
   determining the individual identification information of a relative of the target person by using the second storage unit; and
   acquiring, from the first storage unit, the health information associated with the determined individual identification information, and outputting the acquired health information.

11. The health information output method according to supplementary note 10, wherein
disclosed relative information that determines a relative being capable of disclosing the health information of the individual is defined for each piece of the individual identification information of a plurality of the individuals,
the health information output method further including,
by the computer, determining the disclosed relative information associated with the individual identification information determined by the determination unit, and outputting the health information, when the target person is included in the disclosed relative information.

12. The health information output method according to supplementary note 10 or 11, further including,
by the computer, determining the individual identification information of the relative within a predetermined range.

13. The health information output method according to supplementary note 12, wherein
the relative within the predetermined range is at least one of a parent, a grandparent, a child, a grandchild, and a sibling.

14. The health information output method according to any one of supplementary notes 10 to 13, wherein
disclosure range information indicating a range of the health information disclosable to a relative is defined,
the health information output method further including,
by the computer, defining a range of the health information to be output according to the disclosure range information.

15. The health information output method according to supplementary note 14, wherein a disclosure range of the health information associated with the individual identification information is defined for each of a plurality of pieces of the individual identification information.

16. The health information output method according to any one of supplementary notes 10 to 15, wherein
the health information includes birth information including at least one of progress information when the individual is a fetus, progress information when the individual is born, and progress information when the individual is an infant.

17. The health information output method according to supplementary note 16, further including,
by the computer;
a first sub storage unit that stores medical record information of the individual; and
a second sub storage unit that stores the birth information.

18. The health information output method according to any one of supplementary notes 10 to 17, wherein
the second storage unit is updated according to a birth registration.

19. A program being executable by a computer, wherein
the computer is capable of utilizing a first storage unit that stores, for each of a plurality of individuals, health information of the individual, and individual identification information identifying the individual in association with each other, and is capable of utilizing a second storage unit that stores, for each of a plurality of the individuals, relative information being capable of determining the individual identification information of a relative of the individual,
the program causing the computer to include:
   an acquisition function of acquiring the individual identification information of a target person;
   a determination function of determining the individual identification information of a relative of the target person by using the second storage unit; and
   an output function of acquiring, from the first storage unit, the health information associated with the determined individual identification information, and outputting the acquired health information.

20. The program according to supplementary note 19, wherein
disclosed relative information that determines a relative being capable of disclosing the health information of the individual is defined for each piece of the individual identification information of a plurality of the individuals, and
the output unit determines the disclosed relative information associated with the individual identification information determined by the determination unit, and outputs the health information, when the target person is included in the disclosed relative information.

21. The program according to supplementary note 19 or 20, wherein
the determination unit determines the individual identification information of the relative within a predetermined range.

22. The program according to supplementary note 21, wherein
the relative within the predetermined range is at least one of a parent, a grandparent, a child, a grandchild, and a sibling.

23. The program according to any one of supplementary notes 19 to 22, wherein
disclosure range information indicating a range of the health information disclosable to a relative is defined, and
the output unit defines a range of the health information to be output according to the disclosure range information.

24. The program according to supplementary note 23, wherein
a disclosure range of the health information associated with the individual identification information is defined for each of a plurality of pieces of the individual identification information.

25. The program according to any one of supplementary notes 19 to 24, wherein
the health information includes birth information including at least one of progress information when the individual is a fetus, progress information when the individual is born, and progress information when the individual is an infant.

26. The program according to supplementary note 25, wherein
the first storage unit includes a first sub storage unit that stores medical record information of the individual, and a second sub storage unit that stores the birth information.

27. The program according to any one of supplementary notes 19 to 26, wherein
the second storage unit is updated according to a birth registration.

### REFERENCE SIGNS LIST

10 Health information output apparatus
20 Health information storage unit
22 First sub storage unit
24 Second sub storage unit
30 Relative information storage unit
40 Range information storage unit
50 Terminal
52 Terminal
110 Acquisition unit
120 Determination unit
130 Output unit

What is claimed is:

1. A health information output apparatus comprising:
at least one memory configured to store instructions; and
at least one processor configured to execute the instructions to preform operations comprising:
being capable of utilizing a first storage that stores, for each of a plurality of individuals, health information of the individual, and individual identification information identifying the individual in association with each other,
being capable of utilizing a second storage that stores, for each of a plurality of the individuals, relative information being capable of determining the individual identification information of a relative of the individual,
acquiring the individual identification information of a target person;
determining the individual identification information of a relative of the target person by using the second storage; and
acquiring, from the first storage, the health information associated with the individual identification information determined, and outputting the acquired health information.

2. The health information output apparatus according to claim 1, wherein
disclosed relative information that determines a relative being capable of disclosing the health information of the individual is defined for each piece of the individual identification information of a plurality of the individuals, and
determining the disclosed relative information associated with the individual identification information determined, and outputting the health information, when the target person is included in the disclosed relative information.

3. The health information output apparatus according to claim 1, wherein
determining the individual identification information of the relative within a predetermined range.

4. The health information output apparatus according to claim 3, wherein
the relative within the predetermined range is at least one of a parent, a grandparent, a child, a grandchild, and a sibling.

5. The health information output apparatus according to claim 1, wherein
disclosure range information indicating a range of the health information disclosable to a relative is defined, and
defining a range of the health information to be output according to the disclosure range information.

6. The health information output apparatus according to claim 5, wherein
a disclosure range of the health information associated with the individual identification information is defined for each of a plurality of pieces of the individual identification information.

7. The health information output apparatus according to claim 1, wherein
the health information includes birth information including at least one of progress information when the individual is a fetus, progress information when the individual is born, and progress information when the individual is an infant.

8. The health information output apparatus according to claim 7, wherein
the first storage includes a first sub storage that stores medical record information of the individual, and a second sub storage that stores the birth information.

9. The health information output apparatus according to claim 1, wherein
the second storage is updated according to a birth registration.

10. A health information output method to be performed by a computer, the method comprising,
by the computer;
being capable of utilizing a first storage that stores, for each of a plurality of individuals, health information of the individual, and individual identification information identifying the individual in association with each other, and being capable of utilizing a second storage that stores, for each of a plurality of the individuals, relative information being capable of determining the individual identification information of a relative of the individual;
acquiring the individual identification information of a target person;
determining the individual identification information of a relative of the target person by using the second storage unit; and
acquiring, from the first storage unit, the health information associated with the determined individual identification information, and outputting the acquired health information.

11. A non-transitory computer-readable medium storing a program causing a computer to perform operations comprising:
the computer is capable of utilizing a first storage that stores, for each of a plurality of individuals, health information of the individual, and individual identification information identifying the individual in association with each other, and is capable of utilizing a second storage that stores, for each of a plurality of the individuals, relative information being capable of determining the individual identification information of a relative of the individual,
acquiring the individual identification information of a target person;
determining the individual identification information of a relative of the target person by using the second storage unit; and
acquiring, from the first storage unit, the health information associated with the determined individual identification information, and outputting the acquired health information.
